(19) United States
(12) Patent Application Publication
(10) Pub. No.: US 2008/0208162 A1
(43) Pub. Date: Aug. 28, 2008

(54) Device and Method For Thermophoretic Fluid Delivery

(76) Inventor: Ashok V. Joshi, Salt Lake City, UT (US)

Correspondence Address: CERAMATEC, INC., 2425 SOUTH 900 WEST, SALT LAKE CITY, UT, 84119, US

(21) Appl. No.: 11/845,900

(22) Filed: Aug. 28, 2007

Related U.S. Application Data
(60) Provisional application No. 60/891,712, filed on Feb. 26, 2007.

(51) Int. Cl. A61M 39/00

(52) U.S. Cl. 604/506; 604/291

(57) ABSTRACT

A method and device for thermolphoretic fluid or drug delivery through the creation of a thermal gradient in the skin. The device includes a heat generating mechanism capable of transdermal delivery of a beneficial agent, an insulating portion coupled with the heat generating mechanism, a cold generating mechanism coupled with the insulating portion, and wherein the heat generating mechanism and the cold generating mechanism create a thermal potential across dermal regions. The method includes generating a hot dermal region and transdermally delivering a beneficial agent, insulating, generating a cold dermal region, and creating a thermal potential across the hot dermal region and the cold dermal region.

### RELATED APPLICATIONS

This application is related to and claims the benefit of U.S. Provisional Patent Application Ser. No. 60/891,712 entitled “THERMALPHORETIC FLUID DELIVERY DEVICE AND METHOD” and filed on Feb. 26, 2007 for Ashok V. Joshi, which is incorporated herein by reference.

### FIELD OF THE INVENTION

The present invention relates to a device and method for delivering drugs or other beneficial agents. More specifically, the present invention relates to thermophoretic transport devices and methods of their use in delivering treatment to a body.

### BACKGROUND OF THE INVENTION

Different methods for drug administration have long been known in the practice of medicine. These methods of drug administration include oral consumption of the drug, injections of the drug by needle, and transdermal administration of the drug, etc. The transdermal administration of drugs, beneficial agents, and pharmaceutically active agents is achieved by the direct application of the drug to the skin. The drug is typically applied in such a way that the skin absorbs the drug or beneficial agent thereby taking the drug or beneficial agent into the blood stream.

The dermal administration of drugs features various benefits to a patient including being non-invasive, avoiding metabolism of the drug in the liver, and directed application of the drug to a certain area of the body.

One form of dermal drug administration or delivery is iontophoretic drug delivery. lontophoretic transport of drug or biological treatments is well known, and is commonly used as one way to transport such treatments across a surface and into a body. Many iontophoretic devices have been developed, as witnessed by the quantity of issued patents and pending applications mentioning such phenomena.

Existing iontophoretic devices may generally be classified into two groups based upon their electromotive source. The first such group may be characterized as disposable, and are driven by a galvanic or electrochemical reaction encompassing electrodes bathed in an electrolyte carrying the treatment ions and offering a relatively low voltage. Such devices inherently require long treatment time intervals and are also generally constructed to be inexpensive, used once, and then thrown away. The second type of iontophoretic device typically is driven by an auxiliary power module. While treatment time requirements for devices having auxiliary power modules are generally reduced, the power modules are expensive, and so typically must be reused.

However, iontophoretic devices of both types described above can cause a level of discomfort in the patent depending upon the voltage applied and the sensitivity of the patient. Patients wishing to avoid this discomfort may use a drug delivery patch with no electromotive force. The drawback to this approach is the time required to absorb the medication when no driving force is present. Other limitations include the wide variability of skin permeability among patients, thereby making it nearly impossible to deliver a drug in a consistently timely manner to all types of patients.

One method of non-electrical drug delivery includes a heated patch having a transdermally absorbable drug or beneficial agent. It is known that the application of heat causes the rate of drug absorption to rise. The temperature differential between the heated patch and the dermal region in contact with the patch creates a thermal potential that, much like an electrical or ionic potential, drives the drug or beneficial agent into the dermal region. However, current heated patches are limited by the temperature a patient can comfortably tolerate (about 60 degrees Celsius). The dermal temperature of patients is typically about 37 degrees Celsius, therefore current heat patches are limited to a thermal potential of about 23 degrees Celsius.

What is needed is a simple, disposable, dermal drug or beneficial agent delivery device capable of controllable transdermal drug or beneficial agent delivery and capable of creating a thermal potential in dermal regions.

### BRIEF SUMMARY OF THE INVENTION

The invention provides a method for delivering a treatment to a body by way of an thermophoretic transport procedure and device. A device constructed according to principles of the instant invention provides a low cost, disposable, single use, fast and accurate, thermophoretic fluid delivery device for external or implantable use. A body may be construed specifically as a mammalian (e.g. human or animal) body, or alternatively and generally, as a container of an electrolyte.

An thermophoretic drug delivery device is provided and described herein. In one embodiment, the device includes a heat generating mechanism capable of transdermal delivery of a beneficial agent, and an insulating portion coupled with the heat generating mechanism. The device also includes a cold generating mechanism coupled with the insulating portion, and wherein the heat generating mechanism and the cold generating mechanism create a thermal potential across dermal regions.

In one embodiment, the heat generating mechanism comprises a chemical heat generating mechanism, and the cold generating mechanism comprises a chemical cold generating mechanism. In a further embodiment, the heat generating mechanism and cold generating mechanism are formed from a thermoelectric device comprising a PN junction. The device also includes a heat conductor configured to transfer one of heat and cold to the cold generating mechanism.

In another embodiment, the cold generating device is configured to contract, thereby stretching the heat generating device and subsequently opening pores in a dermal region. Additionally, the device includes a plurality of skin preparation devices coupled with the heat and cold generating mechanisms, and configured to prepare the skin to receive the drug. The skin preparation device may comprise a microneedle configured to penetrate skin. In a further embodiment, the thermal potential between dermal regions comprises a difference in temperature in the range of between about 1 degree Celsius and 75 degrees Celsius.

A method is also provided that includes generating a hot dermal region and transdermally delivering a beneficial agent, insulating, generating a cold dermal region, and creating a thermal potential across the hot dermal region and the cold dermal region. The method may also include transferring one of heat and cold to the cold dermal region, stretching the hot dermal region and increasing transdermal delivery rates of the beneficial agent. Furthermore, the method may include preparing skin to receive the beneficial agent, and penetrating the skin.

Other advantages and aspects of the present invention will become apparent upon reading the following description of the drawings and detailed description of the invention. These and other features and advantages of the present invention will become more fully apparent from the following figures, description, and appended claims, or may be learned by the practice of the invention as set forth hereinafter.

### BRIEF DESCRIPTION OF THE SEVERAL VIEWS OF THE DRAWINGS

In order that the manner in which the above-recited and other features and advantages of the invention are obtained will be readily understood, a more particular description of the invention briefly described above will be rendered by reference to specific embodiments thereof that are illustrated in the appended drawings. Understanding that these drawings depict only typical embodiments of the invention and are not therefore to be considered to be limiting of its scope, the invention will be described and explained with additional specificity and detail through the use of the accompanying drawings in which:

FIG. 1 is a schematic block diagram illustrating one embodiment of a disposable iontophoretic device in accordance with the prior art;

FIG. 2 is a top-view schematic block diagram illustrating one embodiment of a heat patch in accordance with the prior art;

FIG. 3 is a top view schematic block diagram illustrating one embodiment of a thermophoretic device in accordance with the present invention;

FIG. 4 is a cross section diagram illustrating one embodiment of the thermophoretic device in accordance with the present invention;

FIG. 5 is a cross section diagram illustrating one embodiment of a skin preparation device in accordance with the present invention;

FIG. 6 is a top view diagram illustrating one embodiment of the device in accordance with the present invention;

FIG. 7 is a cross section view diagram illustrating an alternative embodiment of the thermophoretic device in accordance with the present invention;

FIG. 8 is a top view diagram illustrating an alternative embodiment of the thermophoretic device in accordance with the present invention; and

FIG. 9 is a schematic flow chart diagram illustrating one embodiment of a method 900 for thermophoretic fluid delivery in accordance with the present invention.

### DETAILED DESCRIPTION OF THE INVENTION

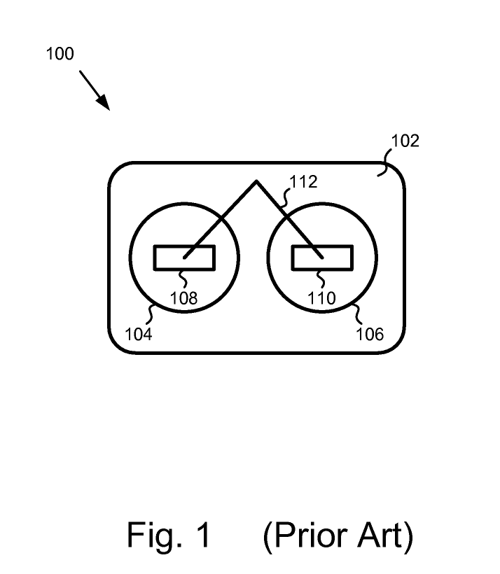

The presented embodiments of the present invention will be best understood by reference to the drawings, wherein like parts are designated by like numerals throughout. It will be readily understood that the components of the present invention, as generally described and illustrated in the figures herein, could be arranged and designed in a wide variety of different configurations. Thus, the following more detailed description of the embodiments of the thermophoretic device of the present invention, as represented in FIGS. 1 through 9, is not intended to limit the scope of the invention, as claimed, but is merely representative of presently preferred embodiments of the invention.

Reference will now be made to the drawings in which the various elements of the invention will be given numerical designations and in which the invention will be discussed so as to enable one skilled in the art to make and use the invention. It is to be understood that the following description is only exemplary of the principles of the present invention, and should not be viewed as narrowing the claims which follow.

FIG. 1 is a schematic block diagram illustrating one embodiment of a disposable iontophoretic device 100 in accordance with the prior art. The disposable iontophoretic device 100 may be constructed on an adhesive strip 102. Cationic chamber 104 and anionic chamber 106 are formed in the adhesive strip 102 to create separated volumes in which to house cationic and anionic treatment materials, respectively. An electrolytic cell created by a chemical reaction between the cationic and anionic electrodes in an electrolyte provides the electromotive force to operate the device for ion transfer to a patient. A first electrode 108 installed in the cationic chamber and a second electrode 110 installed in the anionic chamber are connected by a conductor 112 to form an electron transporting leg of an electric circuit. Application of the adhesive strip to a human body completes the circuit, and initiates a flow of treatment ions through the patient's skin.

An electrode 108 maybe formed from zinc, with an electrode 110 being made from silver chloride. The electrolyte contained in the cationic chamber 104 and anionic chamber 106 directly contacts the skin to be treated, and necessarily is limited in reactivity to avoid skin irritation. Conductive salt solutions (such as 1% NaCl) commonly are employed as electrolytes due to their compatibility with a patient's skin. A device 100, as described, will generate an electromotive force for ion transfer totaling about 1 Volt. In use of a device 100, there is some possibility that a desired treatment chemical may undesirably interact with the electrolyte, electrode, or a product of the galvanic reaction, thereby compromising a treatment.

As described above, the iontophoretic device 100 can be uncomfortable to some people due to the voltage applied to the cathode and the anode. For this reason, pharmaceutically active agents may be applied to non-electrically driven dermal delivery devices. One example of a common non-electrical dermal deliver device is the heat patch.

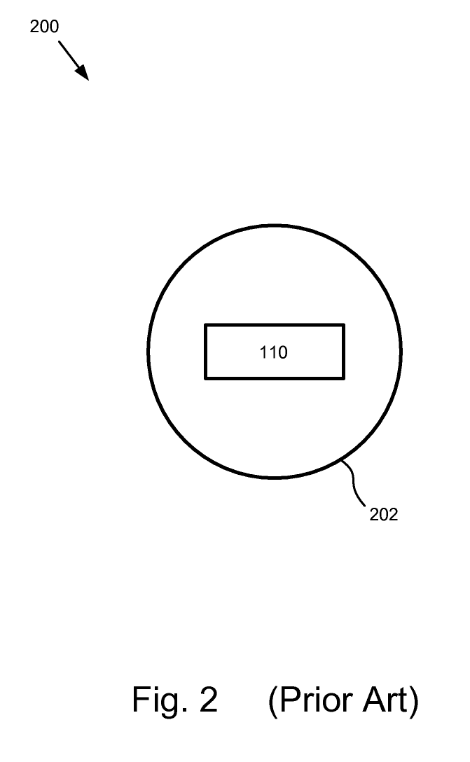

FIG. 2 is a top-view schematic block diagram illustrating one embodiment of a heat patch 200 in accordance with the prior art. The heat patch 200 comprises an adhesive strip 202 having an embedded drug compartment 204. The drug compartment 204 maintains the drug until the heat patch 200 is placed in contact with the skin of a patient. The heat patch, which may be warmed by microwave, hot water, IR, etc., then delivers the drug.

As described above, the heat patch of the prior art is limited by the heat differential between the heat patch and the skin of the patient. The maximum temperature a patient can comfortably tolerate is in the range of about 60-75 degrees Celsius. The average skin temperature of a patient is about 37 degrees Celsius. Therefore, the heat patch is limited to a thermal differential of about 23 degrees Celsius.

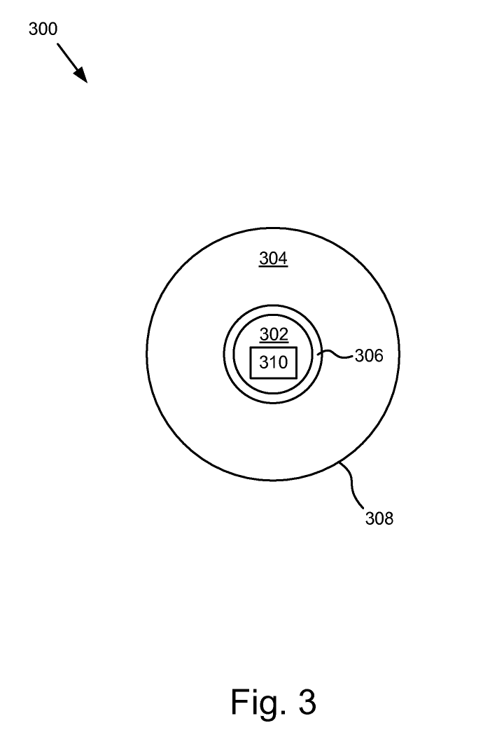

FIG. 3 is a top view schematic block diagram illustrating one embodiment of a thermophoretic device in accordance with the present invention. In one embodiment, the thermophoretic device 300 comprises a heat generating mechanism 302, a cold generating mechanism 304, and an insulating portion 306 disposed between the heat generating mechanism 302 and the cold generating mechanism 304. The thermophoretic device 300 may also include an adhesive base 308 for attaching the thermophoretic device 300 to a patient. This adhesive base may include the adhesive strip 202 described above. The device 300 may include a support structure (discussed in greater detail below) in which the heat generating mechanism 302, the cold generating mechanism 304, and the insulating portion 306 are supported. The adhesive base 308 may be affixed to the support structure. The device 300 may also include a power source (no shown) that may be supported by the support structure. The power source may be direct current, a battery, a galvanic cell, a resistor, and other current generators know in the art.

The thermophoretic device 300 provides a temperature gradient or temperature differential. For example, in one embodiment, the cold generating mechanism can cool to between 0 and 25 degrees Celsius and the heat generating mechanism can heat to between 25 and 75 degrees Celsius. In another embodiment, the cold generating mechanism 204 can cool to between about 0 to 5 degrees Celsius and the heat generating mechanism can heat to between about 50-75 degrees Celsius. Thus, in one embodiment, the thermophoretic device 300 can create a temperature differential of between about 0 to about 75 degrees Celsius. In another embodiment, the temperature differential between the heat generating mechanism 302 and the cold generating mechanism 304 is between about 45 to about 75 degrees Celsius. The thermophoretic device is configured such that the hot and cold generating mechanisms 302, 304 are in communication with the dermal regions on the skin to create temperature differentials in the dermal regions at or beneath the skin surface. The thermal differentials cause a thermal potential, that in a manner similar to electrical potential, drive the delivery of a drug. As used herein, the term “drug” or “beneficial agent” refers to any type of medicament, cosmetic agent, or pharmaceutically active agent or beneficial agent capable of being applied topically or embedded within the skin of a patient. As the drug is delivered, the drug circulates or disperses better as the temperature differential equalizes under thermodynamic principals.

In one embodiment, the thermophoretic device 300 cools the interstitial fluid in the dermal region just beneath the skin while simultaneously heating the drug to be delivered. The resulting temperature gradient allows for improved drug delivery through the skin. As used herein, the term “dermal region” refers to the region of skin beneath hot or cold generating mechanisms. In a further embodiment, a drug delivery compartment 310 may be embedded within the heat generating mechanism 302 and configured to deliver the drug transdermally into the patient.

Heating the dermal region below the heat generating mechanism 302 opens the skin pores adjacent the heat generating compartment to allow for better drug delivery through the skin. Cooling the dermal region beneath the cold generating mechanism 304 causes the pores to constrict. Thus, in one embodiment, the device is configured to create cold skin adjacent heated skin. The cold skin constricts, further pulling on the heated skin and thereby opening the pores even wider, such that the combination provides better skin pore opening at the drug delivery site.

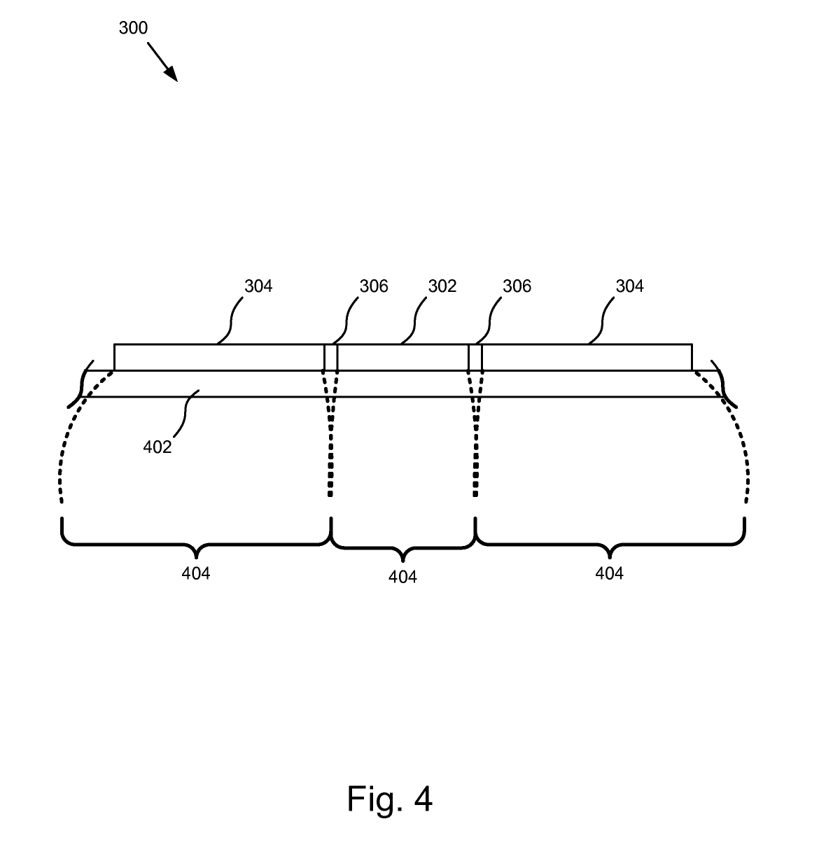

FIG. 4 is a cross section diagram illustrating one embodiment of the thermophoretic device 300 in accordance with the present invention. The thermophoretic device (hereinafter “device”) 300, as described above, comprises a heat generating mechanism 302 and a cold generating mechanism 304 in fluid communication with the skin 402 of a patient. Hot and cold dermal regions 404 are formed beneath the heat generating mechanism 302 and the cold generating mechanism 304, respectively. Drugs absorbed through the skin 402 beneath the heat generating mechanism 302 diffuse to cold dermal regions due to the thermal gradient. Beneficially, the skin absorbs drugs at a faster rate due to the thermal gradient or thermal potential of the dermal regions created by the device 300.

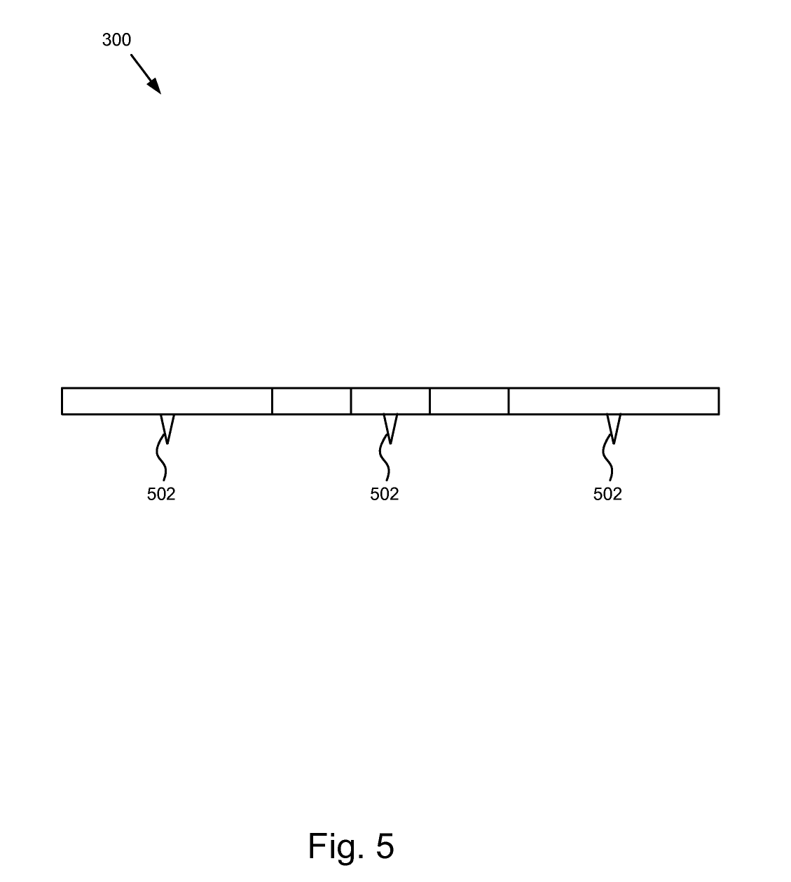

FIG. 5 is a cross section diagram illustrating one embodiment of a skin preparation device in accordance with the present invention. The skin preparation device 502, in one embodiment, comprises a microneedle. The skin preparation device 502 extends downward from the device 300 in order to puncture the surface of the skin or stratum corneum in order to enhance the delivery of the drug into the body. The skin preparation device 502 overcomes the problem of each person having different skin porosity. In one embodiment, the microneedle may have a radius of up to 500 microns and may have a length of up to 3000 microns. The skin preparation device 502 may include an array of microneedles. In one embodiment, an array of microneedles having a radius of 125 microns and a length of 850 microns may be used. It will be appreciated by those of skill in the art that a variety of dimensions and configuration of microneedles may be used to practice the teachings of this invention. The skin preparation device 502 may be supported by the support structure.

In one embodiment, the heat generating mechanism 302 (FIGS. 3 and 4) may include the skin preparation device 502, which may be a microneedle. Similarly, the cold generating mechanism 302 (FIGS. 3 and 4) may include the skin preparation device 502, which may be a microneedle.

Certain people may have such high skin porosity that the device 300 may not be able to effectively deliver the drug into the body. However, once the surface of the skin is broken, the interstitial properties of the body are substantially similar across different races, ages, genders, etc. A consistent porosity enables a health care professional to better gauge the time required to deliver the drug. In a further embodiment, the skin preparation device 502 may comprise a laser, a drill, or any device capable of puncturing, perforating, or making an opening in the skin.

The skin preparation device 502 may be positioned all along the device 300 where the device 300 contacts the skin. In one embodiment, the skin preparation device 502 is positioned adjacent a drug delivery compartment, or alternatively extending outward from the drug delivery compartment. In one embodiment, the drug delivery compartment is embedded within the heat generating compound.

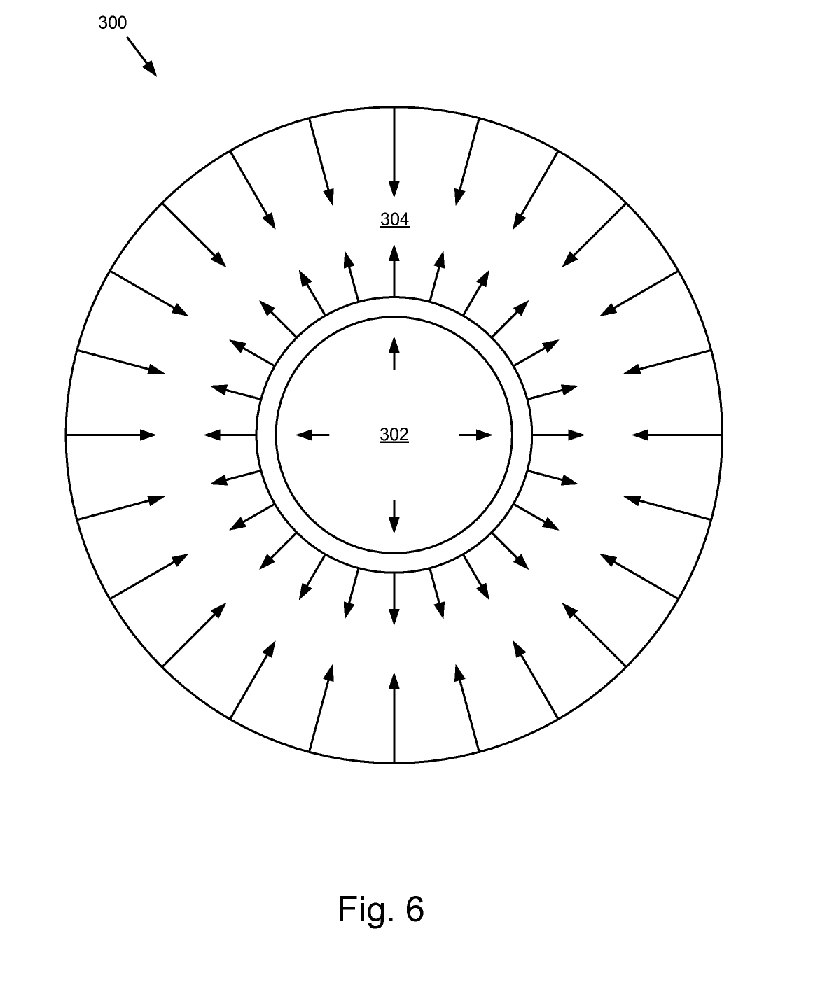

FIG. 6 is a top view diagram illustrating one embodiment of the device 300 in accordance with the present invention. The depicted embodiment comprises a plurality of arrows that indicate the forces the device 300 applies to the skin once the device 300 is activated. The cold generating mechanism 304 contracts as it generates cold. Conversely, the heat generating mechanism 302 expands. As such, the cold generating mechanism 304, together with the heat generating mechanism 302, stretch the dermal region beneath the heat generating mechanism 302 and thereby increase the efficiency of drug delivery.

In one embodiment, the heat generating mechanism 302 may comprise a chemical heater. Examples of chemical heaters capable of being used in the present invention include cellulose, iron, water, activated carbon, vermiculite, salt, and other heaters that produce heat from the exothermic oxidation of iron. Another example of a chemical heater includes exothermic crystallization of supersaturated solutions, such as sodium acetate. These can be recharged by boiling the supersaturated solution and allowing the supersaturated solution to cool. Heating of these chemical heaters can be triggered by snapping a small metal device buried in the heat generating mechanism which generates nucleation points that initiate crystallization. Heat is required to dissolve the salt in its own water of crystallization and it is this heat that is released when crystalisation is initiated.

In a further embodiment, the heat generating mechanism may comprise a chemical heater that uses lighter fluid (lighter fuel) or LPG which is reacted with a platinum catalyst to release heat by oxidation reactions. These can be used on many occasions by simply refueling. It will be appreciated that other sorts of exothermic reactions could be used to heat a node or electrode.

The cold generating mechanism 304 may comprise a device capable of endothermic reactions in order to cool the dermal region adjacent the cold generating mechanism. For example, as with any salt, dissolving ammonium nitrate involves breaking it into its constituent ammonium and nitrate ions, which takes in energy from its surroundings. The formation of new bonds between these ions and surrounding water molecules then releases energy. But since ammonium and nitrate ions are relatively large, the water molecules have relatively weak interactions with their diffuse charges. So with little thermodynamic payback during this bond formation, the immediate effect of adding ammonium nitrate to a solution may be to cool it down. It will be appreciated that any of a number of commercially available chemical coolers may be used to practice the teachings of the invention

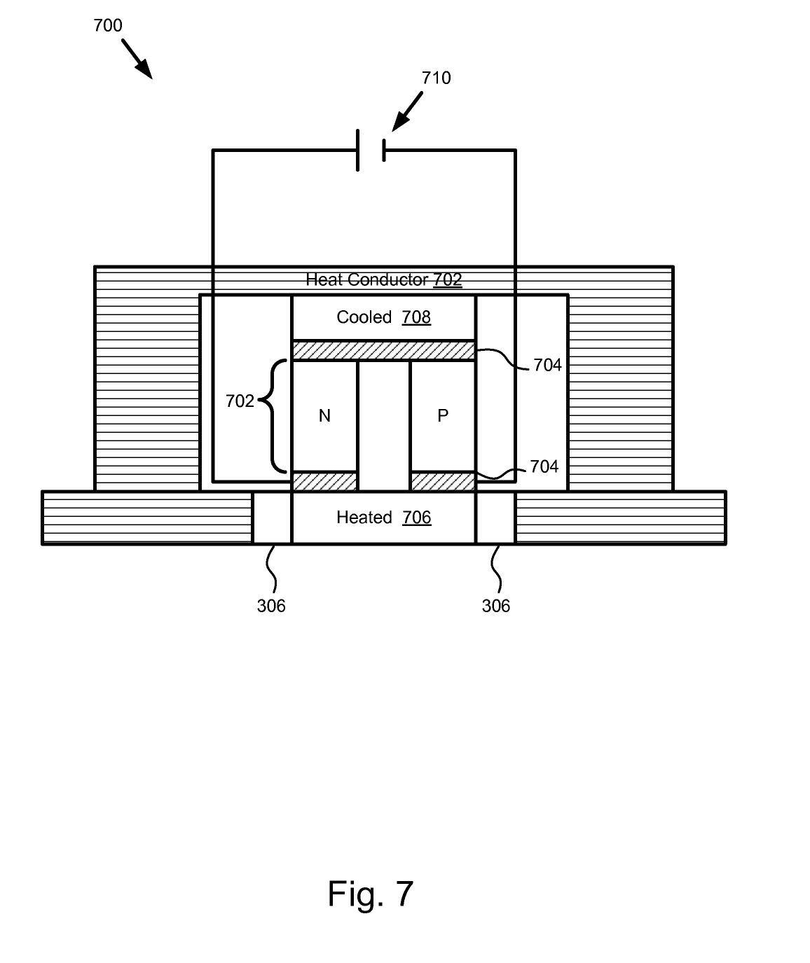

FIG. 7 is a cross section view diagram illustrating an alternative embodiment of the thermophoretic device 700 in accordance with the present invention. In one embodiment the thermophoretic device 700 comprises a heat and cold generating mechanism that comprises a thermoelectric module 702 configured to generate both heat regions and cold regions in the device 700. The thermoelectric module 702, unlike iontophoretic devices, does not pass current or apply a voltage to the skin of the patient. In a further embodiment, the heated and cooled regions 706, 708, may be reversed such that heat is transferred through the heat conductor instead of cold. Thus, the device 700 may be configured where the heat generating mechanisms discussed herein and the cold generating mechanisms discussed herein are a single mechanism coupled to a power source where current direction or polarity can be reversed. In one embodiment, the single mechanism comprises a closed-loop electrical system with a controller.

In a further embodiment, the thermoelectric module 702 comprises a PN semiconductor junction that exhibits the Peltier effect. The Peltier effect occurs when current is passed through dissimilar metals or semiconductors that are connected together at two junctions. The current drives the transfer of heat towards one junction and a subsequent cooling at the other junction. In the depicted embodiment, the junctions 704 transfer the heat to one of either a heated node 706 or a cooled node 708. The device 700 also includes a heat conductor 706 configured to transfer the heat or cold generated by the thermoelectric device 702 to the skin.

With no moving parts, thermoelectric modules are rugged, reliable and quiet heat pumps, typically 1.5 inches (40×40 mm) square or smaller and approximately ¼ inch (4 mm) thick. The industry standard mean time between failures is around 200,000 hours or over 20 years for modules left in the cooling mode. When the appropriate power is applied, from a battery or other DC source, one side of the module will be made cold while the other is made hot. Interestingly, if the polarity or current flow through the module is revered the cold side will become the hot side and vice versa. This allows thermoelectric modules to be used for heating, cooling and temperature stabilization.

Since thermoelectric modules are electrical in nature, in a closed-loop system with an appropriate temperature sensor and controller, thermoelectric modules can easily maintain temperatures that vary by less than one degree Celsius. Simpler on-off control can also be produced with a thermostat. One example of a thermoelectric module capable of being used in the present invention is sold by Advanced Thermoelectric Company, the Melcor Company, and/or the Interface Technology Company. The temperature sensor may be in operable communication with the controller. Furthermore, the on/off switch may be in operable communication with the controller.

It will be appreciated that the nodes could be electrodes or other points capable of transferring temperature to the skin or other surface to be treated. The heat conductor in one embodiment is copper or aluminum. It will be further appreciated by those of skill in the art that various other metals or materials could be used to conduct heat. The drug compartment could be a porous or absorbent pad or material capable of holding a drug to be delivered. For example in one embodiment, the drug compartment is a porous ceramic, in another embodiment, the drug compartment is an absorbent pad, in yet another embodiment, the drug compartment is a container with a membrane. The thermoelectric module 702 may be powered by a battery 710 or other direct current device.

The working life time of such a battery 710 may be controlled to have a desired length by providing only a measured amount of one or more reactant chemicals. The operational life of the battery 710 may be set to last 20 seconds, 20 minutes, or multiple hours, simply by controlling the quantity of reactive components in the battery. Therefore, the effective drug delivery time may be determined in part by the capacity of the battery. Of course, a treatment time may simply be established by operation by a patient, or by a health care practitioner, of a switch to start and stop a flow of current through the device. Total treatment dose may alternatively also be limited by loading a device with a controlled amount of the ion medicament or beneficial agent.

The battery may be manufactured having rugged housings to withstand incidental, or even significant, abuse without incurring sufficient damage to suffer a leak of their contents. For purpose of this disclosure, a battery housing is understood to be rugged if the housing is capable of transferring tissue damaging loads to a patient while avoiding a content leaking rupture. A mini battery having a paper housing, for example, would be susceptible to developing a leak which could harm a patient. Such a paper battery is regarded as not being rugged for purpose of this disclosure.

A familiar example for a rugged battery type is a button-type battery, which is typically housed in a metal canister resembling a button. Such batteries are commonly employed as power sources for wrist watches. A patient wearing a device 700 incorporating such type of rugged battery would be seriously injured before such a metal button battery would leak due to an object contacting the battery. The rugged housing permits safe use of more reactive materials, such as Lithium, Sodium Hydroxide, and Potassium Hydroxide, with correspondingly higher voltage battery outputs than galvanic reactions using low-concentration electrolyte matched to a human body.

In a further embodiment, the thermophoretic device 700 may be combined with an iontophoretic device to enhance drug delivery. Thus, electricity may be applied to the drug compartment side. For example, an electrode may include a drug compartment that is heated either chemically, or by a thermoelectric module. The electrode may be cooled either chemically or by a thermoelectric module. The combination of these two drug delivery methods enhances the efficiency of the drug delivery.

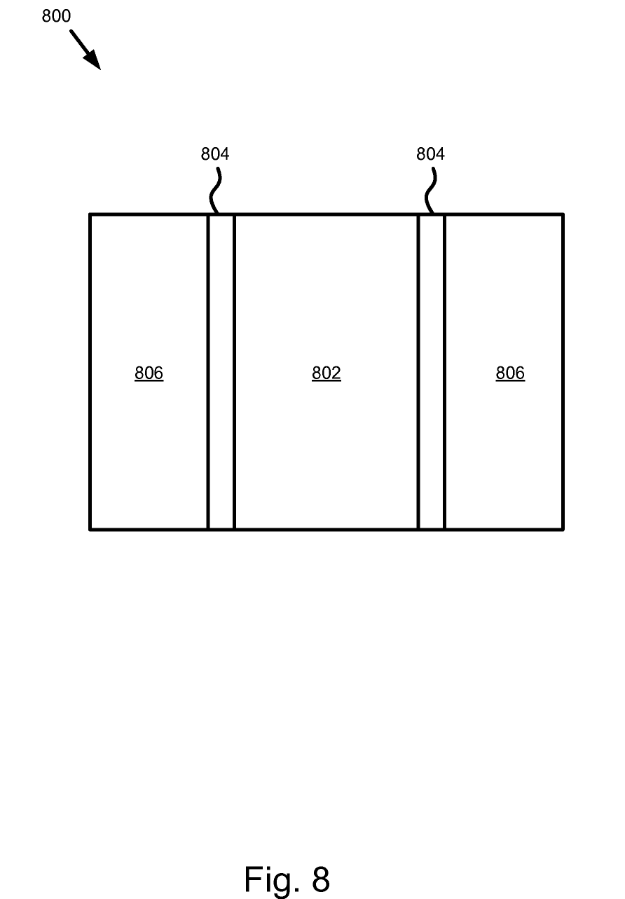

FIG. 8 is a top view diagram illustrating an alternative embodiment of the thermophoretic device in accordance with the present invention. In one embodiment, the device 800 may be configured with a rectangular shape as depicted. The rectangular device 800 comprises a heat generating mechanism 802 coupled with insulating regions 804. Cold generating mechanisms 806 are coupled to the insulating regions 804. As described above with reference to FIG. 6, the cold generating mechanisms 806 will contract and pull the heat generating mechanism outward laterally. The device 800 may also be constructed in any shape deemed suitable for use as a drug delivery patch, such shapes include, but are not limited to, butterfly shapes (for joints, fingers, etc.), ovals, squares, and other geometric shapes.

The schematic flow chart diagram that follows is generally set forth as a logical flow chart diagram. As such, the depicted order and labeled steps are indicative of one embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

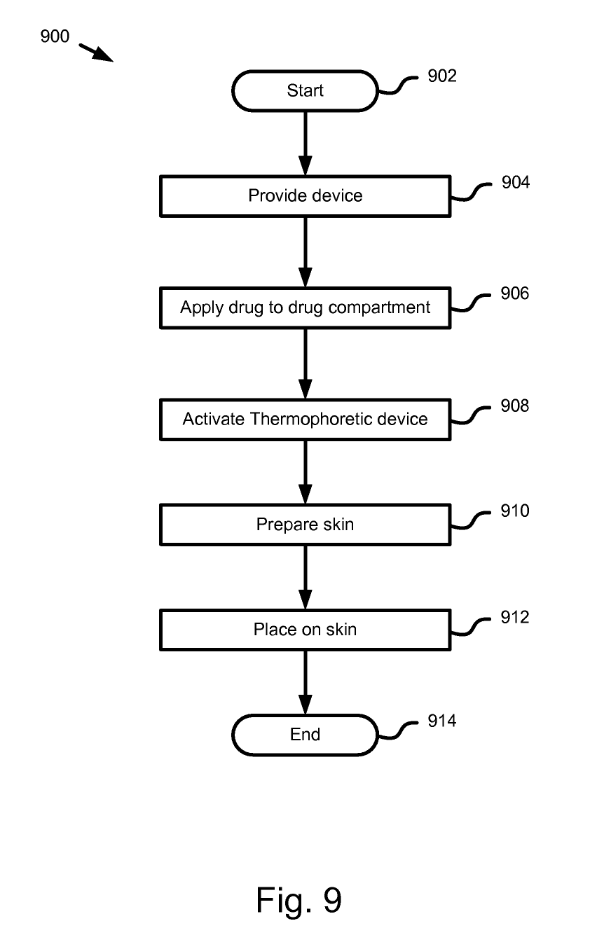

FIG. 9 is a schematic flow chart diagram illustrating one embodiment of a method 900 for thermophoretic fluid delivery in accordance with the present invention. The method 900 starts 902 and a thermophoretic device is provided 904 in accordance with the above described device. The device may be provided with chemical heat and cold generating mechanisms, thermoelectric heat and cold generating mechanisms, or alternatively the device may be heated and cooled using other traditional methods such as an oven warmed heat patch connected to a freezer-cooled cold patch.

The method continues and a health care professional applies 906 a drug to the drug compartment. Alternatively, the drug may be provided at the time of manufacture. The device is then activated 908 and the heat and cold generating mechanisms began to generate heat and cold. In one embodiment, activating the device comprises activating the chemicals as described above with reference to FIGS. 3 and 6. Alternatively, activating the device comprises passing current through the thermoelectric module as described above with reference to FIG. 7.

The patient or heath care provider then prepares the skin 910. In one example, preparing the skin comprises puncturing, perforating, or otherwise preparing a pathway from the surface of the skin to the interstitial fluids of the body beneath the skin. This may include a microneedle puncturing the skin as described above with reference to FIG. 5. The device is then placed 912 on the skin in order to transdermally deliver the drug. Once the drug is delivered the method 900 ends 914.

In another embodiment, a method for thermophoretic fluid delivery may include providing a first and second electrode on a support structure, said electrodes capable of creating a temperature differential. The support structure may be a fabric matrix such as a patch. It may also be gelatinous structure. It will be appreciated by those of skill in the art that the support structure could be a sponge, wicking fibers, fabrics, gauzes, super-absorbent material including super-absorbent polymers that form gels, foams, gelling agents, packing, and other structures an/or substances known to one of ordinary skill in the art. The method may include providing a beneficial agent to the support structure and applying the support structure to a skin surface. The beneficial agent may then be introduced to the skin. In one embodiment, the beneficial agent is delivered with the temperature differential ranging from 5° Celsius to 70° Celsius.

In one embodiment, a method for thermophoretic fluid delivery includes applying a support structure as discussed above that includes a first electrode and a second electrode to a skin surface. The electrodes may be capable of creating a temperature differential at the skin surface. The method may include providing a first beneficial agent to the skin surface adjacent the first electrode at a temperature ranging from 40° Celsius to 75° Celsius and providing a second beneficial agent to the skin surface adjacent the second electrode at a temperature ranging from 5° Celsius to 30° Celsius. The first electrode may include a microneedle and the first beneficial agent may include a beneficial agent selected from a medicinal fluid, a nutritional fluid, a cosmetic fluid, and combinations thereof. The second electrode may also include a microneedle. In one embodiment, the second beneficial agent is or includes water.

While the invention has been described in particular with reference to certain illustrated embodiments, such is not intended to limit the scope of the invention. The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A device for thermophoretic fluid delivery, the device comprising:
a heat generating mechanism capable of transdermal delivery of a beneficial agent;
an insulating portion coupled with the heat generating mechanism;
a cold generating mechanism coupled with the insulating portion; and
wherein the heat generating mechanism and the cold generating mechanism create a thermal potential across dermal regions.

2. The device of claim 1, wherein the heat generating mechanism comprises a chemical heat generating mechanism.

3. The device of claim 1, wherein the cold generating mechanism comprises a chemical cold generating mechanism.

4. The device of claim 1, wherein the heat generating mechanism and cold generating mechanism are formed from a thermoelectric device.

5. The device of claim 4, wherein the thermoelectric device comprises a PN junction.

6. The device of claim 4, further comprising a heat conductor configured to transfer one of heat and cold to the cold generating mechanism.

7. The device of claim 1, wherein the cold generating mechanism is configured to contract, thereby stretching the heat generating mechanism and subsequently opening pores in the dermal region.

8. The device of claim 1, further comprising a plurality of skin preparation devices coupled with the heat and cold generating mechanisms, and configured to prepare the skin to receive the beneficial agent.

9. The device of claim 8, wherein the skin preparation device comprises at least one microneedle configured to penetrate skin.

10. The device of claim 1, wherein the thermal potential between dermal regions comprises a difference in temperature in the range of between about 1 degree Celsius and 75 degrees Celsius.

11. The device of claim 1, wherein the heat generating mechanism comprises a microneedle.

12. The device of claim 1, wherein the cold generating mechanism comprises a microneedle.

13. A device for thermophoretic fluid delivery, the device comprising:
a chemical heat generating mechanism capable of transdermal delivery of a beneficial agent;
an insulating portion coupled with the heat generating mechanism;
a chemical cold generating mechanism coupled with the insulating portion;
wherein the heat generating mechanism and the cold generating mechanism create a thermal potential across dermal regions.

14. The device of claim 13, wherein the cold generating device is configured to contract, thereby stretching the heat generating device and subsequently opening pores in a dermal region.

15. The device of claim 13, further comprising a plurality of skin preparation devices coupled with the heat and cold generating mechanisms, and configured to prepare the skin to receive the beneficial agent.

16. The device of claim 15, wherein the skin preparation device comprises at least one microneedle configured to penetrate skin.

17. The device of claim 13, wherein the thermal potential between dermal regions comprises a difference in temperature in the range of between about 1 degree Celsius and 75 degrees Celsius.

18. A device for thermophoretic fluid delivery, the device comprising:
a thermoelectric heat generating mechanism capable of transdermal delivery of a beneficial agent;
an insulating portion coupled with the heat generating mechanism;
a thermoelectric cold generating mechanism coupled with the insulating portion; and
wherein the heat generating mechanism and the cold generating mechanism create a thermal potential across dermal regions.

19. The device of claim 18, wherein the thermoelectric device comprises a PN junction.

20. The device of claim 18, further comprising a heat conductor configured to transfer one of heat and cold to the cold generating mechanism.

21. The device of claim 18, wherein the cold generating device is configured to contract, thereby stretching the heat generating device and subsequently opening pores in a dermal region.

22. The device of claim 18, further comprising a plurality of skin preparation devices coupled with the heat and cold generating mechanisms, and configured to prepare the skin to receive the beneficial agent.

23. The device of claim 22, wherein the skin preparation device comprises at least one microneedle configured to penetrate skin.

24. The device of claim 18, wherein the thermal potential between dermal regions comprises a difference in temperature in the range of between about 1 degree Celsius and 75 degrees Celsius.

25. A method for thermophoretic fluid delivery, the method comprising:
generating a hot dermal region;
generating a cold dermal region;
creating a thermal potential across the hot dermal region and the cold dermal region; and
transdermally delivering a beneficial agent.

26. The method of claim 25, wherein delivery a beneficial agent comprises transferring beneficial agent from the hot dermal region to the cold dermal region.

27. The method of claim 25, wherein delivery a beneficial agent comprises transferring beneficial agent from the cold dermal region to the hot dermal region.

28. The method of claim 25, further comprising stretching the hot dermal region and increasing transdermal delivery rates of the beneficial agent.

29. The method of claim 25, further comprising preparing skin to receive the beneficial agent.

30. The method of claim 29, further comprising penetrating the skin.

31. A method for fluid delivery, the method comprising:
providing a first and second electrode on a support structure, said electrodes capable of creating a temperature differential;
providing a beneficial agent to the support structure;
applying the support structure to a skin surface; and
introducing the beneficial agent to the skin with said temperature differential ranging from 5° Celsius to 70° Celsius.

32. A method for fluid delivery, the method comprising:
applying a support structure comprising a first electrode and a second electrode to a skin surface, said electrodes capable of creating a temperature differential at the skin surface;
providing a first beneficial agent to the skin surface adjacent the first electrode at a temperature ranging from 40° Celsius to 75° Celsius;
providing a second beneficial agent to the skin surface adjacent the second electrode at a temperature ranging from 5° Celsius to 30° Celsius;

33. The method of claim 32, wherein the first electrode comprises a microneedle.

34. The method of claim 32, wherein the first beneficial agent comprises a beneficial agent selected from a medicinal fluid, a nutritional fluid, a cosmetic fluid, and combinations thereof.

35. The method of claim 32, wherein the second electrode comprises a microneedle.

36. The method of claim 32, wherein the second beneficial agent comprises water.

37. A device for transdermal delivery of a beneficial agent comprising:
a support structure;
a heat generating mechanism supported by the support structure;
a cold generating mechanism supported by the support structure;
an insulating portion disposed between the heat generating mechanism and the cold generating mechanism;
a skin preparation device supported by the support structure;
a drug delivery compartment adjacent one of the heat generating mechanism and the cold generating mechanism; and
wherein the heat generating mechanism and the cold generating mechanism create a thermal potential across dermal regions.

38. The device of claim 37, further comprising an adhesive base.

39. The device of claim 37, wherein the heat generating mechanism comprises a chemical heater.

40. The device of claim 39, wherein the chemical heater comprises a chemical reaction utilizing one component chosen from cellulose, iron, water, activated carbon, vermiculite, salt, and combinations thereof.

41. The device of claim 39, wherein the chemical heater comprises an exothermic crystallization of supersaturated solutions.

42. The device of claim 39, wherein the chemical heater comprises a lighter fuel reacted with a platinum catalyst.

43. The device of claim 37, wherein the cold generating mechanism comprises dissolving ammonium nitrate in an endothermic reaction.

44. The device of claim 37, wherein the heat generating mechanism and the cold generating mechanism is a single mechanism coupled to a power source where current direction can be reversed.

45. The device of claim 44, wherein the single mechanism comprises a closed-loop electrical system with a controller.

46. The device of claim 44, further comprising a temperature sensor in operable communication with the controller.

47. The device of claim 44, further comprising an on/off switch in operable communication with the controller.

48. The device of claim 44, wherein the power source comprises direct current.

49. The device of claim 44, wherein the power source comprises a battery.
